(19) United States
(12) Patent Application Publication
(10) Pub. No.: US 2023/0008386 A1
(43) Pub. Date: Jan. 12, 2023

(54) METHOD FOR AUTOMATICALLY PLANNING A TRAJECTORY FOR A MEDICAL INTERVENTION

(71) Applicant: Quantum Surgical, Montpellier (FR)

(72) Inventors: Bertin NAHUM, Castelnau-le-Lez (FR); Fernand BADANO, Lyon (FR); Lucien BLONDEL, Montpellier (FR)

(21) Appl. No.: 17/757,571

(22) PCT Filed: Dec. 17, 2020
(86) PCT No.: PCT/FR2020/052513

§ 371 Date: Jun. 17, 2022

(30) Foreign Application Priority Data
Dec. 18, 2019 (FR) FR1914780

(51) Int. Cl. A61B 34/10; A61B 34/20; G16H 30/40; G06N 3/08

(52) U.S. Cl. CPC A61B 34/10; A61B 34/20; G16H 30/40; G06N 3/08; A61B 2034/105; A61B 2034/107; A61B 2034/2065

(57) ABSTRACT

The invention relates to a method for automatically planning a trajectory to be followed during a medical intervention by a medical instrument targeting an anatomy of interest of a patient, said automatic planning method comprising the steps of: acquiring at least one medical image of the anatomy of interest; determining a target point on the previously acquired image; generating a set of trajectory planning parameters from the medical image of the anatomy of interest and the previously determined target point, the set of planning parameters comprising coordinates of an entry point on the medical image. The set of parameters is generated using a machine learning method of neural network type. The invention also relates to a guiding device implementing the set of planning parameters obtained.

### TECHNICAL FIELD OF THE INVENTION

The field of the invention is that of assistance in the planning of a medical intervention.

More specifically, the invention relates to a method for automatically planning a trajectory of a medical instrument, to be performed during a medical intervention, and an associated guiding device.

The invention finds applications in particular in the context of a medical intervention during which a medical instrument is inserted into an anatomy of interest, for example to ablate a tumor in an organ, to perform a biopsy, to perform a vertebroplasty or a cementoplasty, or to stimulate an anatomical zone. Such an intervention can optionally be assisted by a medical robot and/or by an augmented reality device.

### PRIOR ART

The prior art has disclosed techniques making it possible to prepare a medical intervention aiming to reach a target anatomical zone in an anatomy of interest of a patient, such as the lungs, kidneys, liver, brain, tibia, knee, vertebra, etc.

Traditionally, the planning of the medical intervention has been carried out manually by an operator on the basis of a medical image obtained by a conventional medical imaging method.

During the planning, the operator defines a target point in the anatomy of interest and an entry point on the patient's skin in proximity to the anatomy of interest, the two points defining a rectilinear trajectory of a medical instrument used during the medical intervention. Such an instrument can be, for example, a needle, a probe or an electrode.

The operator must be attentive to the trajectory that the medical instrument will take, since the trajectory has to respect a number of constraints that are necessary for the smooth conduct of the medical intervention. For example, it may be important that the medical instrument does not pass through bones or blood vessels, especially those with a diameter of more than three millimeters, or that it does not pass through vital organs.

In order to aid the operator in the choice of the entry point in accordance with the target point, planning techniques have been developed in which one or more entry points are automatically proposed to an operator as a function of previously defined constraints, by associating with each corresponding trajectory a score according to predefined criteria.

A technique of this kind is described, for example, in the patent application published under the number US 2017/0148213 A1, entitled “Planning, navigation and simulation systems and methods for minimally invasive therapy”. The method described in said patent application determines trajectories using a conventional image processing algorithm in which the images are segmented in order to be able to minimize constraints relating to the trajectory. For example, during an operation on the brain, the trajectory is determined by an optimization of several parameters, such as minimizing the number of impacted fibers, the distance between a limit of a cortical groove and the target, the volume of white and/or gray matter displaced by the trajectory.

However, the major disadvantage of the techniques in the prior art is that they are generally based on a minimization of constraints that are selected by an operator in order to create a theoretical model, which is often incomplete and imperfect. In addition, they require systematic segmentation of the images in order to be able to optimally calculate the different possible trajectories. This segmentation proves imprecise and incomplete in some cases, which can lead to errors in the trajectory used by the medical instrument.

Furthermore, these techniques do not take into account a possible deformation of the medical instrument, for example a needle, when inserting its end into the body of the patient.

Finally, an experienced operator also intervenes regularly in order to select from the images the regions that are to be avoided, such as blood vessels, and the regions through which the medical instrument must pass, in order to determine the optimal trajectory of the medical instrument.

Interventions by the operator prove tiresome and restrictive, because they require significant attention and experience on the part of the operator in the type of intervention.

None of the current systems makes it possible to simultaneously meet all the required needs, namely to make available an improved technique for automatically planning a medical intervention aimed at reaching a target in an anatomy of interest of a patient, which technique is independent of an operator, while permitting more precise and more reliable planning.

### DISCLOSURE OF THE INVENTION

The present invention aims to overcome all or some of the disadvantages of the prior art mentioned above.

To this end, the invention relates to a method for automatically planning a trajectory to be followed, during a medical intervention, by a medical instrument targeting an anatomy of interest of a patient, said automatic planning method comprising steps of:
acquiring at least one medical image of the anatomy of interest;
determining a target point on the previously acquired image;
generating a set of trajectory planning parameters on the basis of the image of the anatomy of interest and of the previously determined target point, the set of planning parameters comprising coordinates of an entry point on the medical image.

Such a method, used prior to a medical intervention, makes it possible to provide a set of parameters guiding a physician or a surgeon during the manipulation of the medical instrument, which can be a needle, a probe, an electrode or any other medical instrument capable of being inserted into the body of the patient, using a reference point linked to the patient. This reference point is generally three-dimensional in order to guide the medical instrument in space.

The aim of the medical intervention is to reach a target anatomical zone of the body of the patient, for example in order to ablate a tumor in an organ, to perform a biopsy, to perform a vertebroplasty or a cementoplasty, or to stimulate an anatomical zone. The target anatomical zone is situated within or at the surface of an anatomy of interest of the patient. Such an anatomy of interest is, for example, a lung, kidney, liver, tibia, knee, vertebra or brain.

The medical image used for the planning has been obtained, for example, by computed tomography, by magnetic resonance imaging, by ultrasound, by positron emission tomography or by any other medical imaging method.

According to the invention, the set of parameters is generated by implementing an automatic learning method of the neural network type, previously trained on a set of what are called medical training images, each training image comprising an anatomy of interest similar to the anatomy of interest of the patient, each medical training image being associated with coordinates of a target point and of at least one entry point that have been determined beforehand.

Thus, the planning method can be used by any operator, who just has to select a target point on the medical image.

It should be noted that the planning method is based on machine learning of similar medical images, each of them associated with an entry point and a target point.

A similar medical image is understood to mean an image obtained by an identical or equivalent imaging method and comprising the same anatomy of interest in the medical image taken on any individual. It should be noted that the type of medical intervention, the type of medical instrument or the targeted anatomy of interest may be distinct, without prejudice to the precision of the planning parameters obtained. Learning makes it possible in fact to analyze a new image in order to determine an optimal trajectory to the target point chosen by the operator on the medical image of the anatomy of interest of the patient.

It should be noted that the medical training images are generally associated with entry points that are actually used during the medical intervention undergone by the individuals and target points actually reached by the instrument following its insertion. To complete the set of medical training images, medical images associated with assumed entry points, chosen by an operator, can be added to the set.

In addition, the automatic planning method is advantageously based on the learning of non-segmented medical images, that is to say where all or part of the image is characterized according to the type of tissues, organs or vessels present in the part of the image. The processing of the images by the planning method is thus more rapid.

The set of medical training images is generally included in a database or in a medical image bank.

The automatic planning method generally provides planning parameters for at least one possible trajectory. When the automatic planning method provides the planning parameters for several possible trajectories, the operator usually manually selects the trajectory that seems best to him. It should be noted that a trajectory is generally considered to be best when it meets a number of criteria specific to the medical intervention, such as the angle of incidence with respect to a tissue interface (for example the skin, the liver capsule, etc.), the proximity of a blood vessel, organ or bone structure on the trajectory, etc.

It should be noted that the automatic planning method is implemented before any medical, surgical or therapeutic action.

In particular embodiments of the invention, the machine learning method determines the coordinates of the entry point on the basis of the acquired medical image and the target point that is previously determined in the acquired medical image.

In particular embodiments of the invention, the machine learning method firstly generates a probability of being an entry point for each pixel or voxel of the medical image acquired respectively in 2D or in 3D, the coordinates of the entry point corresponding to the coordinates of the pixel or voxel having the greatest probability.

In particular embodiments of the invention, the set of similar medical images comprises a plurality of identical images, each identical image being associated with a distinct point.

Thus, learning is improved because the set of medical images comprises possible trajectory variants for the medical instrument.

Advantageously, the set of similar medical images comprises a plurality of identical images, each identical image being associated with a distinct entry point chosen by a distinct operator.

Thus, the planning parameters obtained are more precise, because they are less sensitive to the choices of a particular operator. It should be noted that the precision of the planning parameters obtained depends on the number of operators involved in analyzing the same medical image during the learning phase.

Preferably, the set of similar medical images comprises at least three identical images, each identical image being associated with a distinct entry point by a distinct operator.

Thus, at least three operators are involved in generating the database comprising the set of medical images that are used during the learning phase.

In particular embodiments of the invention, information relating to the anatomy of interest is associated with each medical image of the set of medical images, the information comprising a type of anatomy of interest or of tumor present in the anatomy of interest, the machine learning method being trained on a number of the set of medical images restricted to the images associated with the same type of anatomy or tumor.

In particular embodiments of the invention, the automatic planning method also comprises a step of allocating a score to a trajectory defined between the entry point of the set of planning parameters and the target point that is determined beforehand on the acquired image.

Thus, the operator is aided in his choice of trajectory from among the possible trajectories provided by the automatic planning method. The score is generally allocated according to criteria that are specific to the medical intervention.

The trajectory defined between the entry point of the set of planning parameters and the target point previously determined on the acquired image is generally rectilinear. However, it can be envisioned that the trajectory is curvilinear, for example substantially along an arc of a circle with a maximum radius of curvature in order to take account of the rigidity of the medical instrument. Generally, a curvilinear trajectory is either concave or convex. In other words, the derivative of a curvilinear trajectory is generally of constant sign, negative or positive, between the entry point and the target point.

Preferably, the allocation of the trajectory score depends on at least one of the following criteria:
the proximity of a blood vessel;
the proximity of an organ;
the proximity of a bone structure;
the angle of incidence with respect to a tissue interface;
the length of the trajectory;
the fragility of a tissue through which the trajectory passes.

In particular embodiments of the invention, the allocation of the trajectory score takes into account a probability of the medical instrument deforming upon contact with a tissue interface.

This deformation generally occurs when the medical instrument has a flexible part, that is to say capable of deforming upon contact with a tissue interface, for example during the insertion of the medical instrument through the skin of the patient.

In particular embodiments of the invention, the allocation of the trajectory score takes into account a recurrence rate or a recovery time associated with a trajectory similar to the planned trajectory.

Thus, the score allocated to the trajectory is negatively impacted if the planned trajectory results in a recurrence rate or a recovery time that is too great or too long for the patient.

In particular embodiments of the invention, the automatic planning method also comprises a step in which the score allocated to the trajectory is compared with a threshold score, the trajectory being validated when the trajectory score is greater than or equal to the threshold score.

In particular embodiments of the invention, the automatic planning method also comprises a step of modifying the entry point when the score allocated to the trajectory is below the threshold score.

In particular embodiments of the invention, the acquired medical image is two-dimensional or three-dimensional.

In particular embodiments of the invention, the medical image is acquired by magnetic resonance, by ultrasound, by computed tomography or by positron emission tomography.

The invention also relates to a device for guiding a medical instrument, comprising means for guiding a medical instrument according to the set of planning parameters obtained by the automatic planning method according to any one of the previous embodiments.

The guiding device can be robotic, a navigation system associated or not associated with a robotic device, an augmented reality device, a patient-specific guide, or a three-dimensional model of the anatomy of the patient.

It should be noted that the device for guiding the medical instrument makes it possible to accompany a practitioner performing the medical intervention.

### BRIEF DESCRIPTION OF THE FIGURES

Other advantages, aims and particular features of the present invention will emerge from the following non-limiting description of at least one particular embodiment of the devices and methods which are the subject matter of the present invention, with reference being made to the accompanying drawings, in which:

FIG. 1 is a schematic view of a medical intervention during which a medical instrument is guided according to a set of parameters established by an automatic planning method according to the invention;

FIG. 2 is a block diagram of an automatic planning method according to a particular embodiment of the invention;

FIG. 3 is an example of a medical image acquired during the first step of the planning method of FIG. 2;

FIG. 4 is an example of a medical image used during the training of the neural network implemented by the method of FIG. 2;

FIG. 5 is a schematic view of a training phase of the neural network implemented by the method of FIG. 2;

FIG. 6 is a schematic view of a development of the neural network implemented by the method of FIG. 2, and trained according to the training phase of FIG. 5;

FIG. 7 is a schematic view of a development of the neural network implemented by the method of FIG. 2, and trained according to an alternative training phase;

FIG. 8 shows two medical images of the same patient, one with a medical instrument inserted and the other corresponding to the same view without the medical instrument, said images being used when learning a neural network configured to define a curvilinear trajectory of a medical instrument.

### DETAILED DESCRIPTION OF THE INVENTION

This description is given without limitation, each feature of an embodiment being able to be combined with any other feature of any other embodiment in an advantageous manner.

It will be noted here that the figures are not to scale.

### Example of a Particular Embodiment

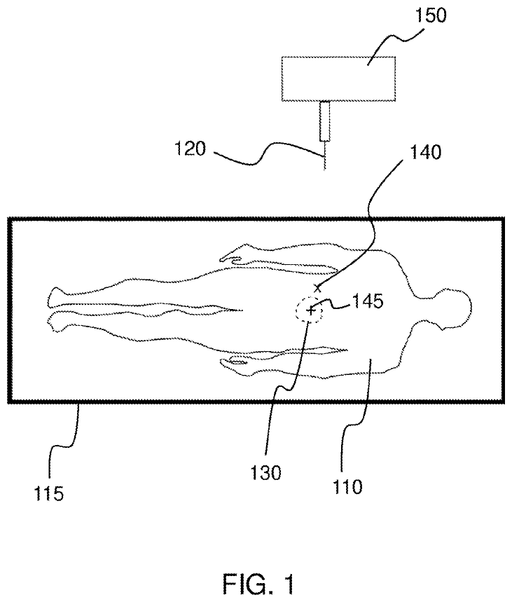

FIG. 1 is a schematic view of a medical intervention during which a patient 110 lying on a table 115 is treated with the aid of a medical instrument 120. In the present non-limiting example of the invention, the medical intervention corresponds to the ablation of a tumor in an anatomy of interest 130, which is here the liver of the patient 110, by way of the medical instrument 120 which is in this case a semi-rigid needle. The medical intervention here is a percutaneous procedure during which the body of the patient 110 is not opened. In addition, the medical intervention can be performed according to different treatment parameters. Such treatment parameters are, for example, a duration and a power of the ablation treatment, a voltage applied in the case of treatment by electroporation, or a frequency applied in the case of treatment by radiofrequency. It should be noted that the present example is given by way of illustration and that a person skilled in the art can implement the invention described below for any type of medical intervention using any medical instrument aimed at an anatomy of interest of the patient.

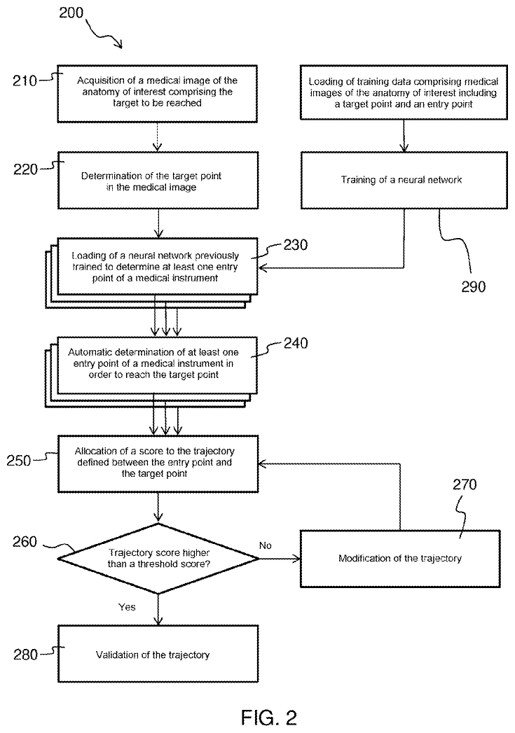

The medical instrument 120 in the present example is advantageously guided by a device 150 along a rectilinear path, by virtue of the prior establishment of a set of planning parameters comprising coordinates of an entry point 140 at the level of the skin of the patient 110, or even an angle to be followed in a three-dimensional reference frame linked to the patient 110 in order to aim at a target point 145 determined beforehand. The set of planning parameters is established by way of an automatic planning method 200 according to the invention, as is illustrated in FIG. 2 in the form of a block diagram.

The method 200 for automatically planning the trajectory to be followed by the medical instrument 120 during the medical intervention comprises a first step 210 of acquiring at least one medical image of the anatomy of interest 130 of the patient 110.

The medical image is generally taken before the medical intervention using equipment dedicated to medical imaging, such as a magnetic resonance imaging (MRI) apparatus, a CT scanner, a spectral scanner or an ultrasound apparatus.

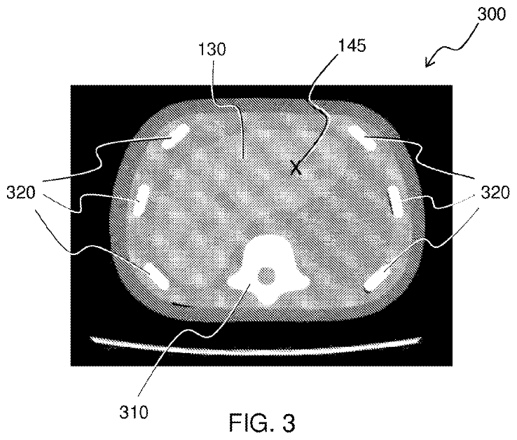

An example of a medical image 300 obtained by computed tomography and showing a model, commonly referred to as a phantom, corresponding to the anatomy of interest 130 of the patient 110 is presented in FIG. 3. The medical image 300 corresponds to a sectional view of the patient 110 according to a plane substantially perpendicular to the axis of the spinal column of the patient 110. In addition to the anatomy of interest 130, the medical image 300 also reveals in particular a vertebra 310 of the spinal column and six ribs 320.

In the previously acquired medical image 300, the target point 145 is determined during a second step 220 of the automatic planning method 200, either manually by an operator or automatically by image analysis.

The target point 145 is associated with coordinates in the medical image 300. These coordinates are two-dimensional or three-dimensional depending on the type of medical image acquired. In the case of a two-dimensional medical image 300, the target point 145 corresponds substantially to one pixel of the image. In the case of a three-dimensional medical image 300, the target point 145 substantially corresponds to one voxel of the image.

In order to determine the coordinates of an entry point of a set of parameters for planning the trajectory to be followed by the medical instrument 120 from the medical image 300 and from the target point 145, a machine learning algorithm, here of the neural network type, is loaded during a third step 230 of the automatic planning method 200.

The neural network has been trained beforehand during a learning phase 290 on a set of medical training images, each of them comprising an anatomy of interest similar to the anatomy of interest 130. The medical training images have generally been acquired on a cohort of individuals, each medical training image being associated with coordinates of a target point and of an entry point that have been previously determined generally by at least one operator.

Advantageously, the set of medical training images comprises several times the same medical image, but associated with distinct entry points generally determined by at least three operators.

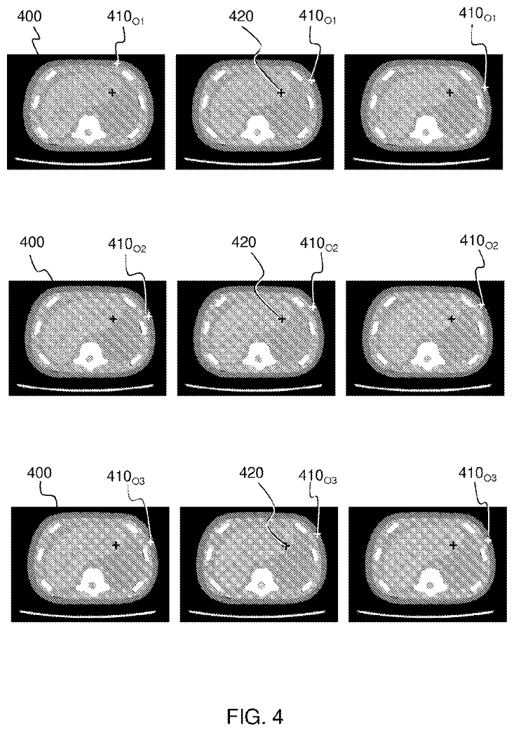

FIG. 4 shows an example of the same medical image 400 comprising each time the same target point 420. This medical image 400, included nine times in the set of medical training images, has been processed by three separate operators 01, 02 and 03, who have each provided three entry points, respectively 410₀₁, 410₀₂ and 410₀₃.

The training of the neural network can be advantageously restricted to the images associated with a given item of information, such as the type of anatomy of interest or of the tumor present in the anatomy of interest, in order to increase the consistency by decreasing the variability of the sets of planning parameters that the neural network can obtain.

It should be noted that there may be hardware limitations to training a neural network, especially when the set of medical training images comprises three-dimensional images of the anatomy of interest. In order to overcome these hardware limitations, it is possible to reduce the resolution of each medical image, but with the risk of reducing the precision of the parameters obtained by the neural network. It is also possible to restrict the training to the trajectories parallel to a predetermined plane, such as a plane perpendicular to the axis of the spinal column of the patient. Another solution to overcome the hardware limitations can be to use chips commonly referred to as tensor processor units, which are dedicated to machine learning.

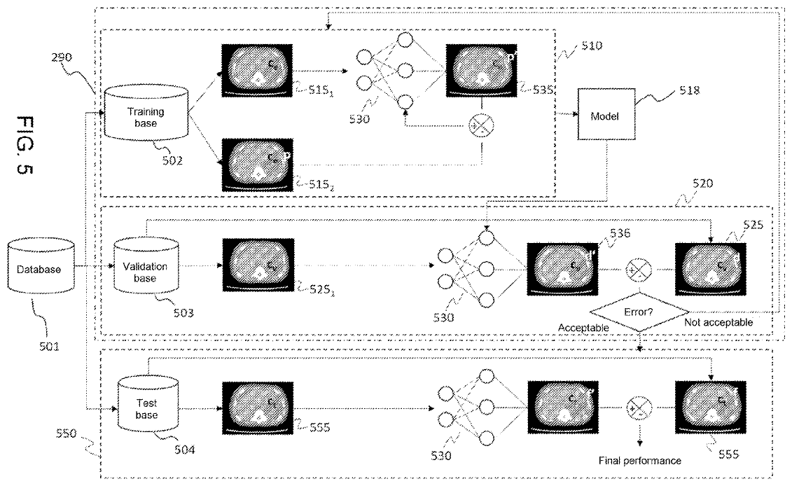

The phase 290 of training the neural network, as is illustrated in more detail in FIG. 5, generally comprises two main steps 510, 520, which can be repeated, and requires a database 501 comprising a set of medical images where each image is associated with an entry point and with a target point. Optionally, information on the properties of the instrument used to perform the intervention, such as the length of the instrument or the coefficient of stiffness of the instrument, is also associated with each medical image of the database 501. After the training phase 290, a possible test phase 550 can be implemented.

The database 501 of medical images is divided into three databases 502, 503, 504 comprising distinct medical images. The three databases 502, 503, 504 are called the training base, the validation base and the test base, respectively.

In the present non-limiting example of the invention, 60 to 98% of the medical images of the database 501 are grouped together in the training base 502, 1 to 20% in the validation base 503, and 1 to 20% in the test base 504. The percentages, generally functions of the number of images in the database 501, are given here by way of indication.

During the first step 510 of the training phase, medical images 515 of the training base 502 are used to determine a weight W and a bias b for each neuron of the neural network 530 that is used to obtain the coordinates of the entry point of the set of trajectory planning parameters.

To determine the weight W and the bias b of each neuron, each medical image 515 of the training base 502 is proposed to the neural network 530 according to two variants, a first 515₁ comprising only the target point cₑ, and a second 515₂ comprising both the target point cₑ and the predetermined entry point p. From the first variant of the medical image 515₁, the neural network 530 then makes a prediction 535 on the position of the entry point p′. The coordinates of the predicted entry point p′ are compared with the coordinates of the position of the predetermined entry point p, associated with the second variant of the medical image 515₂. The error between the coordinates of the predicted entry point p′ and the predetermined entry point p is then used to adjust the parameters W and b of each neuron of the neural network 530. A model 518 is obtained at the end of the first step 510 of the training phase.

During the second step 520 of the training phase, the medical images 525 of the validation base 503, advantageously distinct from the medical images 515, are used to validate the weight W and the bias b of each neuron of the neural network 530.

During this second step 520 of the training phase 290, a variant 525₁ of each medical image comprising only the position of a target point cᵥ is proposed to the neural network 530. The neural network 530 then makes a prediction 536 on the position of the entry point d′. The coordinates of the predicted entry point d′ are compared with the coordinates of the position of the predetermined entry point d, associated with the medical image 525 used for validation. The error between the coordinates of the predicted entry point d′ and of the predetermined entry point d is then used to verify the parameters W and b of each neuron of the neural network 530 that are determined in the first step 510.

In the case where the prediction error of the neural network would be too great at the end of this second step 520, the neural network 530 is re-trained according to the two steps 510 and 520 of the training phase 290 previously described, by reusing the same medical training images 515 and validation images 525.

Alternatively, during the re-training of the neural network 530, the first step 510 uses all or some of the validation images 525. The second step 520 of re-training the neural network uses as many training images 515 as there are validation images 525 used for the first step 510 of re-training.

It should be noted that the neural network 530 can be re-trained as many times as is necessary to reduce the prediction error.

When the two steps 510, 520 of the training phase 290 are implemented at least once, the final performance of the neural network can be tested during a possible test phase 550 with the medical images 555 of the test base 504. These medical images 555, advantageously distinct from the images 515 and 525, make it possible to verify that the neural network 530 as configured with the parameters W and b for each neuron makes it possible to predict with good precision the coordinates of an entry point in all the situations with which the neural network 530 is likely to be confronted. A comparison is thus made between the coordinates of the entry point f′, as predicted by the neural network 530, and the predetermined entry point f in the so-called test medical image 555. This comparison is identical to the one carried out during the second step 520 of the training phase. However, in contrast to step 520, this test phase 550 does not result in a new training cycle of the neural network 530. If the performance of the neural network 530 is not good at the end of the step 550, the training phase 290 is then recommenced with a new untrained neural network.

It should be noted that the images 555 used in the test phase 550 are generally carefully selected so as to cover different positions of the target point cₜ in the anatomy of interest, in order to optimally test the prediction capabilities of the training network 530.

In an alternative training phase, the neural network can be trained to provide, for each pixel or voxel of a medical image, a probability that actually corresponds to the entry point. The set of medical images used for this alternative training can be identical to the set of medical images used previously. However, it may be preferable, for this alternative training, to use medical images having several entry points on the same image. Advantageously, the entry points displayed on the same image are determined by at least three distinct operators. The alternative training of the neural network takes place in three steps similar to the training phase described above.

The previously trained neural network makes it possible to determine, during the fourth step 240 of the automatic planning method 200, at least one set of parameters for planning the trajectory to be followed by the medical instrument 120 on the basis of the analysis.

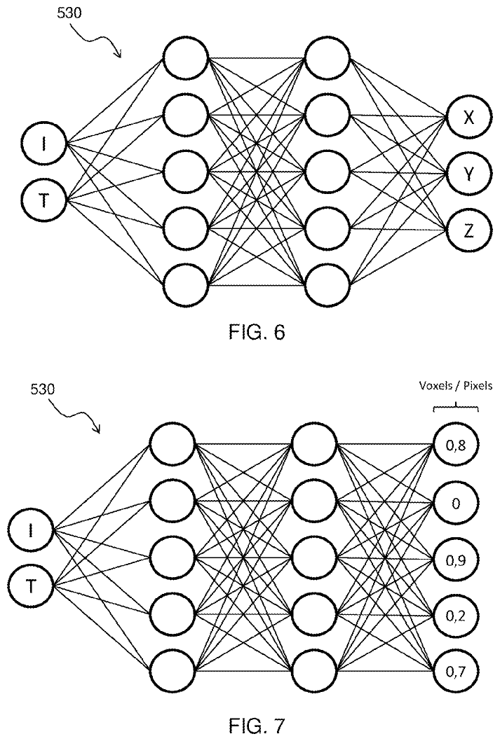

In the case where the neural network is trained according to the training phase 290, the neural network 530 will provide, from the medical image I and from the coordinates of the target point T, three-dimensional coordinates (x, y, z) of the entry point in the acquired medical image, as is illustrated in FIG. 6.

In the case where the neural network is trained according to the alternative training phase, the neural network 530 will provide, from the medical image I and from the coordinates of the target point T, a probability, for each pixel or voxel of the medical image, of being the entry point, as is illustrated in FIG. 7. The pixel or voxel having the highest probability is then selected as being the entry point.

The automatic planning method 200 illustrated in FIG. 2 comprises a fifth step 250 implemented when a trajectory is determined by means of a set of planning parameters that is generated by the neural network. During this fifth step 250, a score is allocated to the trajectory defined by the straight line connecting the entry point and the target point.

For example, the score allocated to the trajectory is between 0 and 100, the score of 100 corresponding to the score of an ideal trajectory.

In variants of this particular embodiment of the invention, the trajectory is curvilinear, obtained for example by calculating the most probable trajectory on the acquired medical image, previously segmented, or by a neural network having previously learnt the trajectories that are followed during earlier medical interventions by a similar or identical medical instrument, in particular in terms of stiffness and length. The set of parameters then comprises additional parameters making it possible to define the predicted trajectory between the entry point and the target point.

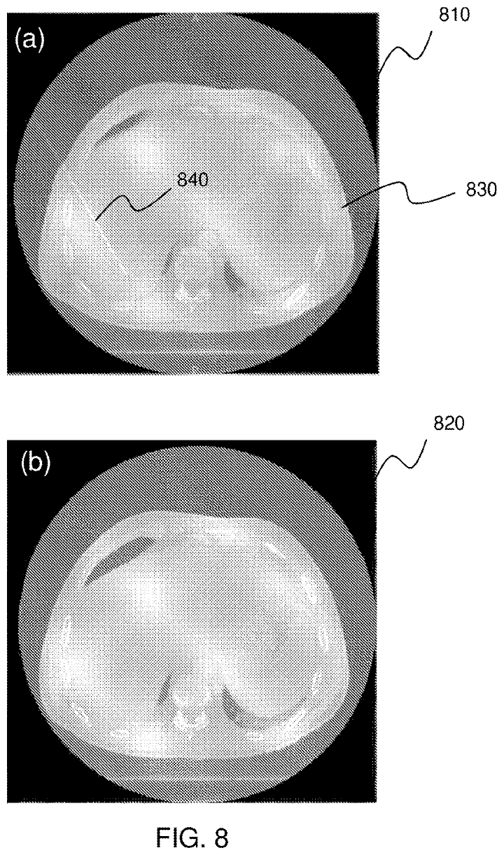

By way of illustration of these alternative embodiments of the invention, FIG. 8 shows two medical images 810, 820 of a patient 830 with or without a medical instrument 840. By making a difference between the two medical images 810 and 820, it is possible to determine the trajectory actually taken by the medical instrument 8840. This trajectory can also be determined by carrying out a recognition of the medical instrument 840 in the medical image 810, for example by detecting strong variations in intensity or contrasts at the pixels/voxels of the medical image 810, in order to route the medical instrument 840 in the medical image 810.

The trajectory score is generally determined on the basis of criteria that can be ranked in order of importance. It should be noted that the examples of criteria described below are not limiting and that other criteria specific to a given medical intervention can be used to determine the trajectory score.

The trajectory score can be calculated, for example, as a function of the proximity of the trajectory to a blood vessel. This is because when the trajectory of the medical instrument is likely to pass through a blood vessel, there is a risk that bleeding will occur. Therefore, the greater the number of blood vessels present on the trajectory, the lower the score allocated to the trajectory.

It should be noted that the size of a blood vessel can be taken into account in this evaluation of the score. For example, if a blood vessel with a diameter of greater than or equal to 3 mm is situated on or near the trajectory calculated by the neural network, points are automatically deducted from the score, for example 50 points on the scale from 0 to 100, because these blood vessels can be vital to the patient. When a blood vessel that is passed through proves to be a vena cava, a portal vein or the aorta, the score is automatically equal to 0, which may be the case in particular when removing a tumor from the liver.

The trajectory score can also be calculated according to the proximity of the trajectory to an organ and/or a bone structure.

In fact, for some interventions, for example on soft tissue, there must be no bone structure situated on the trajectory. If there is, the score allocated to the trajectory is zero.

For other interventions, for example on bone structures such as a knee or a shoulder, passing through a bone structure does not negatively impact the allocated score. More precisely, if the trajectory passes through a predetermined bone structure, the allocated score may be increased.

With regard to organs, the trajectory score is generally reduced when an organ at risk, such as a lung, the intestine or a muscle, is situated at least in proximity to the trajectory. This is also the case when a nerve, a bile duct, a ligament, a tendon or a neighboring organ of the anatomy of interest is situated at least in proximity to the trajectory.

The trajectory score can also be calculated according to the angle of incidence of the trajectory with a tissue interface at the entry point.

For example, in the case of insertion of a semi-rigid needle along a trajectory tangential to a tissue interface, such as the skin or the liver capsule, there is a risk of the needle bending and not following the planned trajectory. The smaller the angle between the trajectory and the tissue interface, the lower the trajectory score. The criterion may be reflected by the fact that the optimal trajectory corresponds to an angle, between the tissue interface and the trajectory, of greater than 20°.

The trajectory score can also be calculated according to the angle of incidence of the trajectory with a bone structure.

For example, in the case of an intervention on a bone structure, there is a risk of the medical instrument slipping on the bone when it is inserted tangentially to the bone. The criterion is then reflected by the fact that the greater the angle between the trajectory and the bone structure, the lower the trajectory score.

The trajectory score can also be calculated according to the length of the trajectory, so as to minimize the length of the trajectory and the inherent risk of causing damage in the patient's body.

The trajectory score can also be calculated according to the fragility of a tissue that is passed through.

For example, in the particular case of an intervention on a patient's brain, the trajectory score can be reduced if the planned trajectory passes through fragile tissues.

In the case of insertion of a semi-rigid needle, the score can also be calculated according to a probability of deformation of the needle during the insertion. This probability is calculated using information on the type of needle used, such as the length, the coefficient of stiffness, or the shape of the bevel of the needle, combined with the information previously determined, i.e. the type of tissue passed through, the angle of incidence and/or the length of the trajectory.

It should be noted that, in order to calculate the trajectory score, the acquired medical image may have been segmented beforehand so as to identify the different types of elements present in the acquired image, such as tissue, a blood vessel, a bone structure, etc., and situated on or in proximity to the trajectory defined between the predicted entry point and the predetermined target point. This segmentation of the acquired image is used only when the trajectory of the medical instrument is generated, and not during the generation of the trajectory by the neural network.

The trajectory score, obtained according to the criteria specific to the medical intervention, can be weighted as a function of a recurrence rate and/or with a recovery time.

As regards the recurrence rate, the score obtained is reduced when the trajectory planned by the neural network is similar to a trajectory which was used with the same treatment parameters during previous medical interventions using the same medical instrument, and for which the recurrence rate of the individuals having undergone these medical interventions is notable.

Likewise, as regards the recovery time, the score obtained is reduced when the recovery time observed previously for individuals having undergone a medical intervention with a trajectory similar to the planned trajectory is long, for example greater than three days.

The score allocated to the planned trajectory is then compared with a threshold score during a sixth step 260 of the automatic planning method 200.

For example, on a scale of 0 to 100, the planned trajectory can be validated only if the score allocated is greater than or equal to 50. Preferably, the planned trajectory is validated if its score is greater than or equal to 70.

In the case where the score allocated to the trajectory is less than the threshold score, the operator has the option of manually modifying the entry point during a possible seventh step 270 of the automatic planning method 200. The modification is carried out, for example, via a graphical interface until the modified trajectory score is greater than the threshold score.

Alternatively, the trajectory can be modified automatically using a gradient algorithm, a graph algorithm, or any other optimization algorithm (Momentum, Nesterov Momentum, AdaGrad, RMSProp, Adam, etc.).

Finally, when the score for the trajectory provided by the neural network, possibly modified, is greater than or equal to the threshold score, the trajectory is validated during an eighth step 280 of the automatic planning method 200.

The validated trajectory can then be used during the medical intervention in order to guide the insertion of the medical instrument 120 into the anatomy of interest 130 of the patient 110 with very good precision and with the greatest chance of the medical intervention going well.

It should be noted that the reference frame used for the guiding generally corresponds to the table 115 on which the patient 110 is lying. The coordinates of the target point are advantageously transferred to the guide reference frame in which characteristic points of the patient 110 have been calibrated beforehand. This operation of transfer and calibration of the guide reference frame is common.

The guiding device 150 can then be used to guide the medical instrument 120 by following the set of planning parameters of the validated trajectory.

The guiding device 150 may be robotic, a navigation system associated or not associated with a robotic device, an augmented reality device, a patient-specific guide 110, or a three-dimensional model of the anatomy of the patient 110.

The augmented reality device can be, for example, a pair of glasses in which the planned trajectory is projected onto at least one of the lenses of the pair of glasses. The augmented reality device can also be a screen placed in proximity to the patient 110, the screen displaying the planned trajectory. The augmented reality device can also comprise a projector projecting the planned trajectory onto the body of the patient 110 or can be a holographic device.

The guiding device can comprise optical navigation means, electromagnetic navigation means or an inertial unit having acceleration and rotation sensors.

The set of planning parameters of the validated trajectory can be used to construct a patient-specific guide 110. This specific guide is generally used in the context of a medical intervention such as open surgery on a bone structure. It should be noted that the patient-specific guide is a personalized, single-use medical device, generally 3D printed. The patient-specific guide helps prevent inaccuracies during surgery, in order to perform an intervention as planned. The specific guide generally matches the shape of the bone structure corresponding to the anatomy of interest and makes it possible to guide the insertion of a medical instrument according to the orientation of the planned trajectory.

In the case of some medical interventions, a three-dimensional model of the anatomy of the patient 110, commonly referred to as a phantom, is constructed in order to permit training prior to the medical intervention. The three-dimensional model of the anatomy of the patient 110 can then advantageously comprise an indication of the entry point of the medical instrument as defined in the set of planning parameters of the trajectory obtained by the automatic planning method 200.

The results obtained by the automatic planning method 200 can also be used to present the medical intervention to peers, for example physicians, surgeons or radiologists, or even to the patient 110 who is to undergo the medical intervention. These results can also be used to train peers in the medical intervention.

1. A method for automatically planning a trajectory to be followed during a medical intervention by a medical instrument targeting an anatomy of interest of a patient, said automatic planning method comprising the steps of:
acquiring at least one medical image of the anatomy of interest;
determining a target point on the previously acquired image; and
generating a set of trajectory planning parameters from the medical image of the anatomy of interest and from the previously determined target point, the set of planning parameters comprising coordinates of an entry point on the medical image;
wherein the set of parameters is generated using a machine learning method of the neural network type, previously trained on a set of medical training images, each medical training image comprising an anatomy of interest similar to the anatomy of interest of the patient, each medical training image being associated with coordinates of a target point and of at least one entry point that have been previously determined.

2. The automatic planning method of claim 1, wherein the machine learning method determines the coordinates of the entry point from the acquired medical image and from the target point previously determined in the acquired medical image.

3. The automatic planning method of claim 1, wherein the machine learning method first generates a probability of being an entry point for each pixel or voxel of the medical image acquired in 2D or 3D respectively, the coordinates of the entry point corresponding to the coordinates of the pixel or voxel having the greatest probability.

4. The automatic planning method of claim 1, wherein the set of similar medical images comprises a plurality of identical images, each identical image being associated with a distinct entry point.

5. The automatic planning method of claim 1, wherein the set of similar medical images comprises a plurality of identical images, each identical image being associated with a distinct entry point chosen by a distinct operator.

6. The automatic planning method of claim 1, wherein information relating to the anatomy of interest is associated with each medical image of the set of medical images, the information comprising a type of anatomy of interest or tumor present in the anatomy of interest, the machine learning method being trained on a number of the set of medical images restricted to the images associated with the same type of anatomy or tumor.

7. The automatic planning method of claim 1, further comprising a step of allocating a score to a trajectory defined between the entry point of the set of planning parameters and the target point previously determined on the acquired image.

8. The automatic planning method of claim 7, wherein the acquired image is mapped, the allocation of the trajectory score being a function of at least one of the following criteria:
the proximity of a blood vessel;
the proximity of an organ;
the proximity of a bone structure;
the angle of incidence with respect to a tissue interface;
the length of the trajectory; or
the fragility of a tissue through which the trajectory passes.

9. The automatic planning method of claim 7, wherein the allocation of the trajectory score takes into account a probability of the medical instrument deforming upon contact with a tissue interface.

10. The automatic planning method of claim 7, wherein the allocation of the trajectory score takes into account a recurrence rate associated with a trajectory similar to the planned trajectory.

11. The automatic planning method of claim 7, wherein the allocation of the trajectory score takes into account a recovery time associated with a trajectory similar to the planned trajectory.

12. The automatic planning method of claim 7, further comprising a step in which the score allocated to the trajectory is compared with a threshold score, the trajectory being validated when the trajectory score is greater than or equal to the threshold score.

13. The automatic planning method of claim 7, further comprising a step of modifying the entry point when the score allocated to the trajectory is below the threshold score.

14. A device for guiding a medical instrument, comprising means for guiding a medical instrument according to the set of planning parameters obtained by the automatic planning method of claim 1.

15. The guiding device of claim 14, being either a robotic guiding device, a navigation system associated or not associated with a robotic device, an augmented reality device, a patient-specific guide, or a three-dimensional model of the anatomy of the patient.
